(19) United States
(12) Patent Application Publication
(10) Pub. No.: US 2018/0343949 A1
(43) Pub. Date: Dec. 6, 2018

(54) Cap with Memory Foam

(71) Applicant: Steven Cunliffe, Rancho Santa Fe, CA (US)

(72) Inventor: Steven Cunliffe, Rancho Santa Fe, CA (US)

(21) Appl. No.: 15/997,017

(22) Filed: Jun. 4, 2018

Related U.S. Application Data
(60) Provisional application No. 62/515,103, filed on Jun. 5, 2017.

(51) Int. Cl. A42B 1/00; A42B 1/22; A42B 7/00; A61M 21/00

(52) U.S. Cl. CPC A42B 1/006; A42B 1/22; A42B 7/00; A61M 2205/0266; A61M 2021/0016; A61M 2209/088; A61M 21/00

(57) ABSTRACT

This invention provides a cap with a superior comfort feature. The cap teaches a sweat band with a pellon attaching the band to the cap, a layer of memory foam, and a layer of wicking fabric. The goal behind the cap is to wick away perspiration from the user and at the same time enhance the comfort of the fit with memory foam. The memory foam ban extends approximately 280 degrees around the inside of the cap, thereby providing support and comfort on all sides of the user's head. The memory foam can be infused with a variety of substances, ranging from biologically-reactive chemicals to aromatherapy agents.

### CROSS REFERENCE TO RELATED APPLICATIONS

This application claims priority to U.S. Provisional Application No. 62/515,103, entitled “Cap with Memory Foam” filed 5 Jun.2017.

### STATEMENT REGARDING FEDERALLY SPONSORED RESEARCH OR DEVELOPMENT

This invention was not federally sponsored.

### INVENTOR

Cunliffe, Stephen E., resident of USA, citizen of USA.

### BACKGROUND OF THE INVENTION

### Field of the invention

This invention relates to the general field of caps, and more specifically toward a cap with an inner band with memory foam.

### Statement of the Problem Solved.

The invention provides a comfortable cap that has additional benefits for the user through the inclusion of various substances into a memory foam layer disposed around the inside of the cap.

### Prior Art

There is considerable prior art that attempts to make caps comfortable for the wearer. Some have used elastic to make the diameter of the cap fit a variety of heads; others have used soft material to line the band around the inside of the cap to make it more comfortable for the wearer. Still other cap manufacturers have placed different types of liners at different parts of the band with the hope that the different materials will provide a different degree of support for the various parts of the head.

However, none of the prior art offers a solution that is both comfortable and gives the manufacturer of the cap the option of making a variety of caps—all identically constructed in terms of the shape and size of the components—but with different additives that give the cap different properties. For example, the memory foam in this cap can be infused with substances that can do everything from removing odors to treating mood swings through aromatherapy. It is also conceived that the memory gel that is infused into the foam making up the center of the band could be selectively infused in different densities, thereby creating a cap with different degrees of memory in different portions of the cap.

### SUMMARY OF THE INVENTION

The cap provides a number of improvements over the prior art. First, it has a band of memory foam extending around the entire inside band of the cap other than the closure. Second, it has an inner wicking fabric designed to pull moisture away from the head of the user, and a pellon, or back that attaches the band to the rim of the cap. The wicking fabric and pellon can be made from a number of materials, including but not limited to nylon webbing, elastic and plastic. The wicking fabric and pellon can be, optionally, stretchable on non-stretchable, biaxially stretchable or unidirectionally stretchable.

Third, is has multiple stitching patterns contemplated to keep the memory foam layer relatively flat. Fourth, the memory foam can be infused with one or more additional chemicals or substances, thereby providing a secondary (or more) function to the cap.

Among the additive substances contemplated are those designed to reduce odors include but are not limited to Aloe Vera, green tea extract and activated charcoal. The wicking fabric can be created from a variety of materials as well, including but not limited to other foams, rayon, cotton, and even phase-change materials (PCM's).

### OBJECTS OF THE INVENTION

It is a principal object of the invention to provide a cap that is comfortable for the user.

It is another object of the invention to provide a cap with memory foam completely surrounding the head of the user along the bottom back of the cap other than the closure element.

A further object of the invention is to provide a cap with memory foam infused with at least one additional compound.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims.

### BRIEF DESCRIPTION OF THE FIGURES

The accompanying drawings, which are incorporated in and form a part of this specification, illustrate embodiments of the invention and together with the description, serve to explain the principles of this invention.

### REFERENCE NUMBERS USED

1. Band, generally.

2. Closure, generally

3. Wicking fabric

4. Memory Foam

5. Pellon

6. Stitching pattern

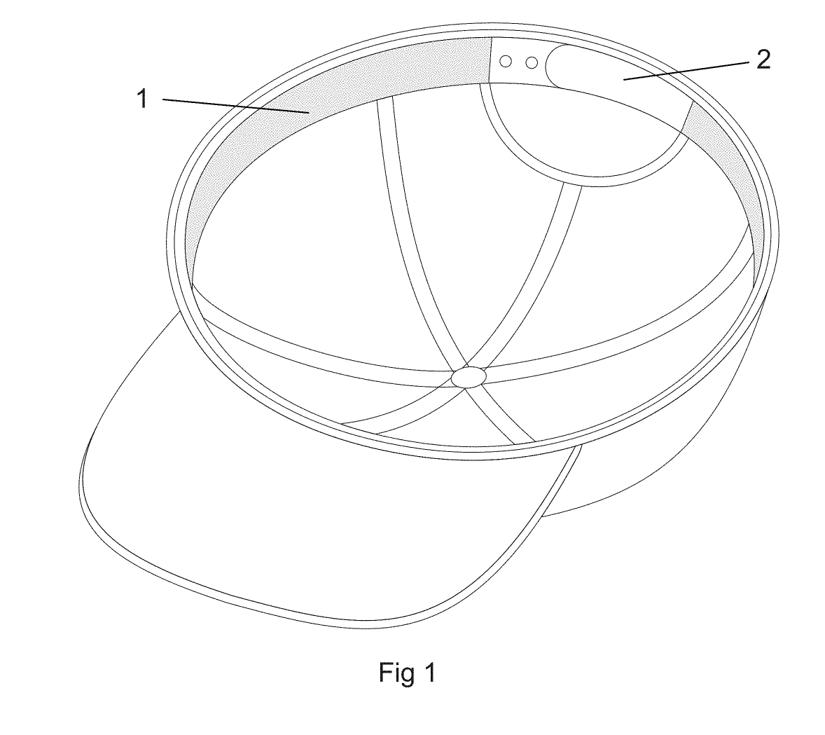

FIG. 1 is a bottom, perspective view of a cap.

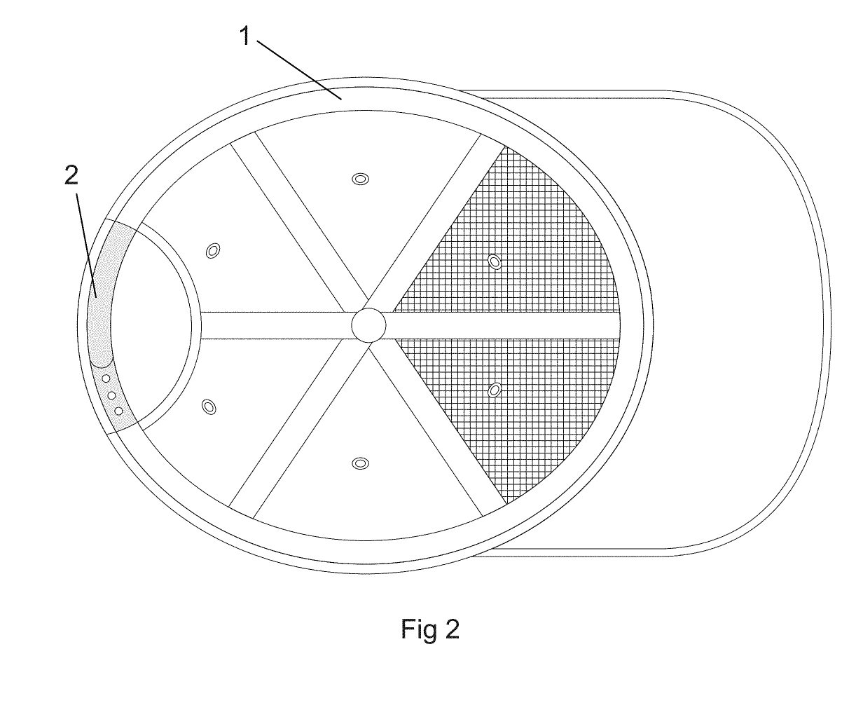

FIG. 2 is another bottom, perspective view of a cap.

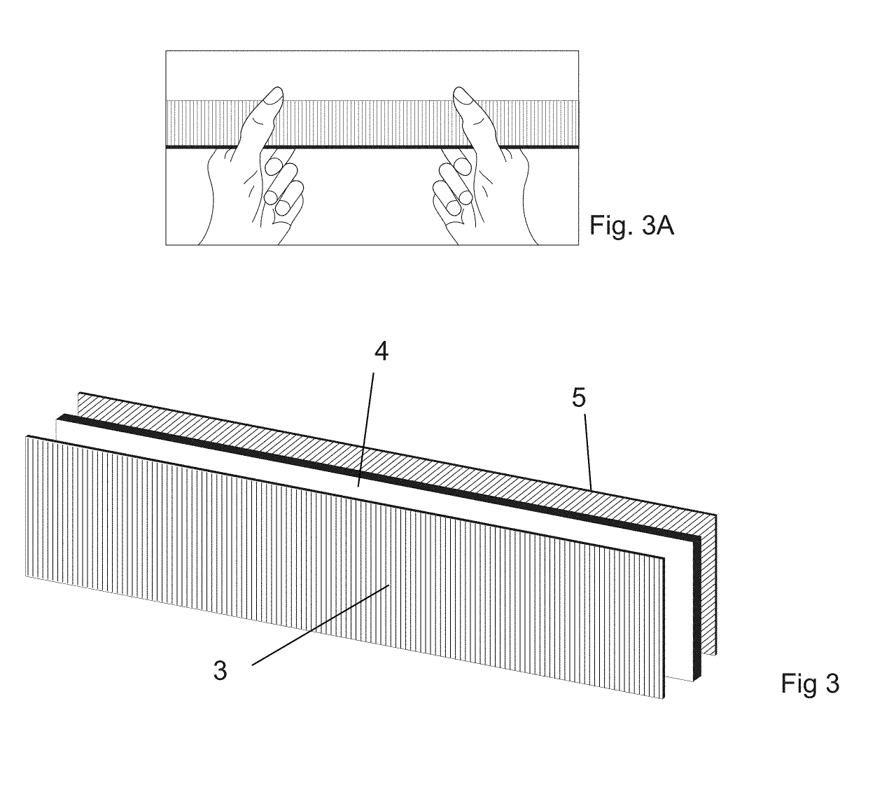

FIG. 3 is a side, perspective view of the band portion of the cap.

FIG. 3A is a front view of a person holding the band

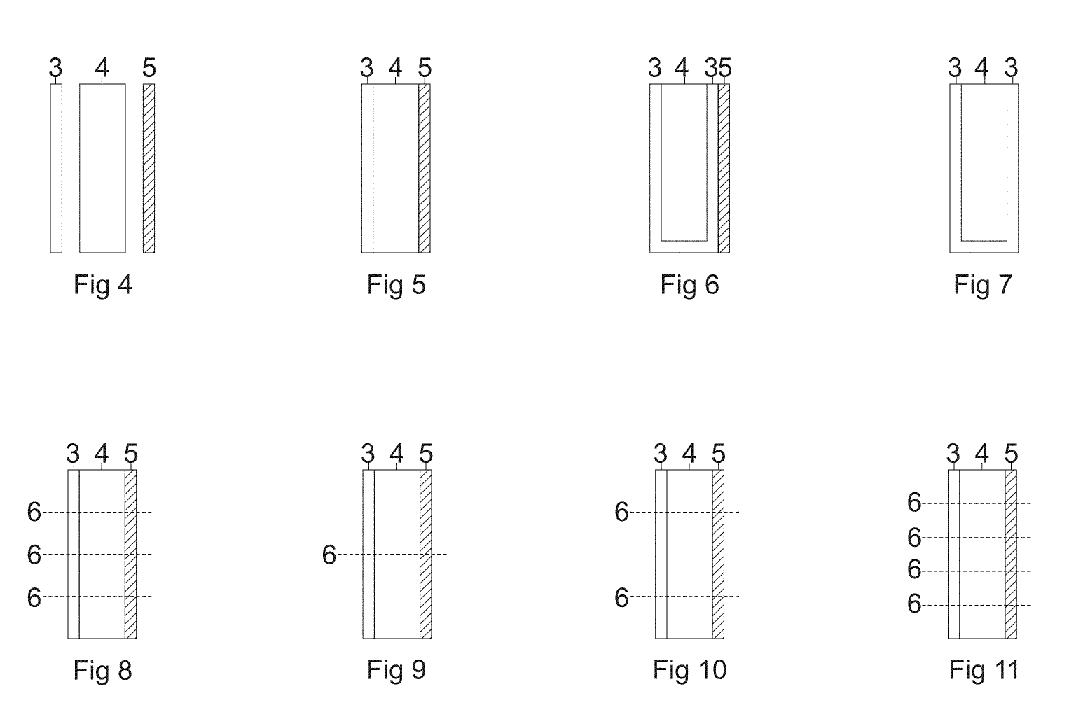

FIGS. 4-7 are a series of cross-sectional view of various embodiments of the band.

FIGS. 8-11 are different views of how the stitching pattern can keep the band relatively flat.

### DETAILED DESCRIPTION OF THE INVENTION

Many aspects of the invention can be better understood with the references made to the drawings below. The components in the drawings are not necessarily drawn to scale. Instead, emphasis is placed upon clearly illustrating the components of the present invention. Moreover, like reference numerals designate corresponding parts through the several views in the drawings.

FIG. 1 is a bottom, perspective view of a cap. The band 1 extends around the entire circumference of the cap other than for the closure 2 section.

FIG. 2 is another bottom, perspective view of a cap. The band 1 extends around the entire circumference of the cap other than for the closure 2 section.

FIG. 3 is a side, perspective view of the band portion of the cap along with a front view of a person holding the band. The wicking fabric 3 rests against the head of the user, pulling away sweat. The memory foam layer 4, absorbs the water through the wicking fabric 3, and retains memory of the head configuration of the user. The pellon 5 is the backer of the band and connects the band to the inner edge of the cap. The band is created from these three materials and, as can be seen in the FIG. 3A, which is a front view of a person holding the band.

FIGS. 4-7 are a series of cross-sectional view of various embodiments of the band. FIG. 4 shows a cross-sectional view of the three basic components lined up for adhesion. FIG. 5 is cross-sectional view of the components as shown in FIG. 4 after they have been adhered to each other. FIG. 6 is a slightly different configuration, where the wicking fabric 3 extends over the front, bottom, and side of the memory foam 4, with the pellon 5 still serving as the backer. FIG. 7 is a different embodiment of the invention, where the wicking fabric 3 serves as both “front” and “backer” for the memory foam 4.

FIGS. 8-11 are different views of how the stitching pattern 6 can keep the band relatively flat. Because the memory foam is porous and spongy by nature, it is, optionally, desirable to restrain the outward expansion of the memory foam through one or more lines is stitching 6.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

What I claim is:

1. A cap with a sweat band disposed around the inner bottom of the cap, where, the cap has an inner perimeter, where the inner perimeter comprises a comfort section and a closure section, and the sweat band is disposed around all of the comfort section, where the sweat band comprises a layer of wicking fabric, a layer of memory foam, and a layer of pellon, where the memory foam additionally comprises a quantity of gel, where the comfort section is disposed over 280 degrees of the inner perimeter, where the gel has been infused into the memory foam at the time of the creation of the memory foam.

2. The cap of claim 1, where the gel is infused into the memory foam with at least two densities, a first density memory foam and a second density memory foam, where the at least two densities are different from one other and provide a first density supportive and memory feature and a second density supportive and memory feature, and where the first density supportive and memory feature is different from the second density supportive and memory feature.

3. The cap of claim 2, where at least one of the first density memory foam and the second density memory foam additionally comprises an additive, where the additive is an odor-reducing additive.

4. The cap of claim 3, where the odor-reducing additive is selected from the group consisting of Aloe Vera, green tea extract and activated charcoal.

5. The cap of claim 2, at least one of the first density memory foam and the second density memory foam additionally comprises an additive where the additive has aromatherapy qualities.

6. The cap of claim 5, where the additive with aromatherapy qualities is selected from the group consisting of lavender, chamomile and cinnamon.

7. The cap of claim 2, where the wicking fabric is selected from the group consisting of foams, rayon, cotton, and even phase-change materials (PCM's).

8. The cap of claim 7, where the memory foam has a front and a back, and the wicking fabric extends across both the front and the back of the memory foam, and where the pellon attaches the sweat band to the cap.

9. The cap of claim 8, where the wicking fabric is selected from the group consisting of fabric, nylon webbing, elastic and plastic.

10. The cap of claim 9, where the pellon is selected from the group consisting of fabric, nylon webbing, elastic and plastic.

11. A cap with a sweat band disposed around the inner bottom of the cap, where the cap has an inner perimeter, where the inner perimeter comprises a comfort section and a closure section, and the sweat band is disposed around all of the comfort section for a closure length of approximately 280 degrees of the inner perimeter, where, the sweat band comprises a layer of wicking fabric and a layer of memory foam.

12. The cap of claim 11, additionally comprising a layer of pellon.

13. The cap of claim 12, where the memory foam additionally comprises a quantity of gel.

14. The cap of claim 13, where the gel has been infused into the memory foam at the time of the creation of the memory foam.

15. The cap of claim 14, The cap of claim 1, where the gel is infused into the memory foam with at least two densities, a first density memory foam and a second density memory foam, where the at least two densities are different from one other and provide a first density supportive and memory feature and a second density supportive and memory feature, and where the first density supportive and memory feature is different from the second density supportive and memory feature.

16. The cap of claim 15, where the memory foam additionally comprises an additive.

17. The cap of claim 16, where the additive is an odor-reducing additive selected from the group consisting of Aloe Vera, green tea extract and activated charcoal.

18. The cap of claim 17, where the memory foam comprises a second additive, where the second additive has aromatherapy qualities and is selected from the group consisting of lavender, chamomile and cinnamon.

19. The cap of claim 18, where the wicking fabric is selected from the group consisting of foams, rayon, cotton, and even phase-change materials (PCM's), and, where the memory foam has a front and a back, and the wicking fabric extends across both the front and the back of the memory foam, and where the pellon attaches the sweat band to the cap.

20. The cap of claim 19, where the wicking fabric is selected from the group consisting of fabric, nylon webbing, elastic and plastic, where the pellon is selected from the group consisting of fabric, nylon webbing, elastic and plastic, and where the closure section is selected from the group consisting of hook-and-loop fasteners, buckles, and snaps.
